(19) United States
(12) Patent Application Publication
(10) Pub. No.: US 2011/0160310 A1
(43) Pub. Date: Jun. 30, 2011

(54) TCB BASED HYDROPHILIC POLYURETHANE DISPERSIONS

(75) Inventors: Jürgen Köcher, Langenfeld (DE); Christian Wamprecht, Neuss (DE)

(73) Assignee: Bayer MaterialScience AG, Leverkusen (DE)

(21) Appl. No.: 13/061,338

(22) PCT Filed: Aug. 22, 2009
(86) PCT No.: PCT/EP2009/006101

§ 371 Date: Feb. 28, 2011

(30) Foreign Application Priority Data
Sep. 4, 2008 (EP) 08015573.2

(51) Int. Cl. A61K 47/34; C08L 75/12; A61P 7/02

(52) U.S. Cl. 514/772.3; 524/591; 524/500

(57) ABSTRACT

The present invention relates to a polyurethaneurea dispersion including a polyurethaneurea which is terminated with at least one polyethylene oxide- and polypropylene oxide-based copolymer unit, and includes a polycarbonate polyol-based unit of formula (I)

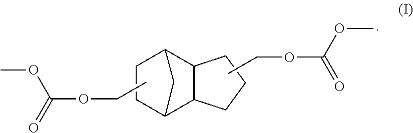

The present invention relates to innovative aqueous polyurethane dispersions which can be used for producing hydrophilic coatings.

Putting hydrophilic surfaces on medical devices such as catheters for example may cause their use to be greatly improved. The insertion and displacement of urinary or blood-vessel catheters is made easier by the fact that hydrophilic surfaces in contact with blood or urine adsorb a film of water. This reduces the friction between the catheter surface and the vessel walls, and so the catheter is easier to insert and move. Direct watering of the devices prior to the intervention can also be performed in order to reduce friction through the formation of a homogeneous water film. The patients concerned experience less pain and the risk of injuries to the vessel walls is reduced by such measures. Furthermore, when catheters are being used in contact with blood, there is always the risk of formation of blood clots. In this context, hydrophilic coatings are generally considered to be useful for antithrombogenic coatings.

Suitable in principle for producing such surfaces are polyurethane coatings which are produced starting from solutions or dispersions of corresponding polyurethanes.

For instance, U.S. Pat. No. 5,589,563 describes the use of coatings having surface-modified end groups for polymers that are used in the biomedical sector, and these coatings can also be used to coat medical devices. The resulting coatings are produced starting from solutions or dispersions, and the polymeric coatings comprise different end groups, selected from amines, fluorinated alkanols, polydimethylsiloxanes and amine-terminated polyethylene oxides. As a coating for medical devices, however, these polymers do not have satisfactory properties, particularly as regards the required hydrophilicity.

DE 199 14 882 A1 relates to polyurethanes, polyurethane-ureas and polyureas in dispersed or dissolved form which are synthesized from
a) at least one polyol component,
b) at least one di-, tri- and/or polyisocyanate component,
c) at least one hydrophilic, nonionic or potentially ionic synthesis component composed of compounds having at least one group that is reactive towards isocyanate groups and having at least one hydrophilic polyether chain, and/or of compounds having at least one group which is capable of forming salts and is optionally in at least partially neutralized form, and having at least one group that is reactive towards isocyanate groups,
d) at least one synthesis component from the molecular weight range 32 to 500 that is different from a) to c) and has at least one group that is reactive towards isocyanate groups, and
e) at least one monofunctional blocking agent. The polymer dispersions that hence necessarily have a monofunctional blocking agent are used in sizes.

DE 199 14 885 A1 relates to dispersions based on polyurethanes, polyurethane-polyureas and polyureas that are preferably reaction products of
a) at least one polyol component,
b) at least one di-, tri- and/or polyisocyanate component,
c) if desired, at least one (potentially) ionic synthesis component composed of compounds having at least one group that is reactive towards NCO groups and having at least one group that is capable of forming salts and is optionally in at least partially neutralized form,
d) if desired, at least one nonionically hydrophilic synthesis component, composed of compounds which are monofunctional to tetrafunctional with respect to the isocyanate addition reaction and contain at least one hydrophilic polyether chain,
e) if desired, at least one synthesis component from the molecular weight range 32 to 2500 that is different from a) to d) and has groups that are reactive towards isocyanate groups, and
f) 0.1% to 15% by weight of at least one monofunctional blocking agent of which at least 50% is composed of dimethylpyrazole,
   the sum of a) to f) being 100%, and where either c) or d) cannot be 0 and are employed in an amount such that a stable dispersion is formed. The dispersions are put to uses including the coating of mineral substrates, the varnishing and sealing of wood and wood-based materials, the painting and coating of metallic surfaces, the painting and coating of plastics and the coating of textiles and leather.

These prior-art polyurethaneurea dispersions are not used for medical purposes, i.e., for coating medical devices. Furthermore, the existing polyurethaneurea coatings frequently have disadvantages in that they are insufficiently hydrophilic for use as a coating on medical devices.

In this context U.S. Pat. No. 5,589,563 recommends surface-modified end groups for biomedical polymers that can be used to coat medical devices. These polymers comprise different end groups, which are selected from amines, fluorinated alkanols, polydimethylsiloxanes and amine-terminated polyethylene oxides. As a coating for medical devices, however, these polymers likewise do not have satisfactory properties, particularly as regards the required hydrophilicity.

European Application No. 08153053.7, unpublished at the priority date of the present specification, then discloses aqueous dispersions which can be used outstandingly for producing hydrophilic coatings.

It has now been found that the mechanical properties of such coatings can be improved further by using specific polycarbonate diols.

The invention accordingly provides polyurethaneurea dispersions comprising polyurethaneureas which
(1) are terminated with at least one polyethylene oxide- and polypropylene oxide-based copolymer unit, and
(2) comprise polycarbonate polyol-based units of formula (I)

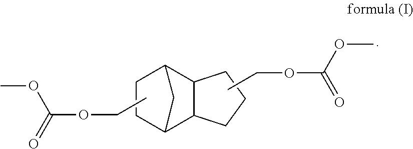

In accordance with the invention it has been found that compositions comprising these specific polyurethaneureas are outstandingly suitable as hydrophilic coatings, for medical devices among others, to which they give an outstanding lubricious coating and at the same time reduce the risk of the formation of blood clots during treatment with the medical device.

Polyurethaneureas for the purposes of the present invention are polymeric compounds which have

a) at least two repeating units containing urethane groups, of the following general structure

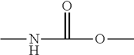

and
b) at least one repeating unit containing urea groups

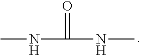

The dispersions according to the invention are based on polyurethaneureas which have substantially no ionic modification. By this is meant, in the context of the present invention, that the polyurethaneureas for use in accordance with the invention have substantially no ionic groups, such as, in particular, no sulphonate, carboxylate, phosphate and phosphonate groups.

The term “substantially no ionic modification” means, in the context of the present invention, that ionic modification is present in a fraction of not more than 2.50% by weight, preferably not more than 2.00% by weight, in particular not more than 1.50% by weight, more preferably not more than 1.00% by weight, especially not more than 0.50% by weight, it being most preferred that there is no ionic modification at all of the polyurethaneurea provided in accordance with the invention.

The polyurethaneureas of the aforementioned kind that are essential to the invention are preferably substantially linear molecules, but may also be branched, although this is less preferred. Substantially linear molecules in the context of the present invention are systems with low levels of incipient crosslinking, the parent polycarbonate polyol component having an average hydroxyl functionality of preferably 1.7 to 2.3, more preferably 1.8 to 2.2, very preferably 1.9 to 2.1. Such systems can still be sufficiently dispersed.

The number-average molecular weight of the polyurethaneureas that are essential to the invention is preferably 1000 to 200 000 g/mol, more preferably from 5000 to 100 000 g/mol. This number-average molecular weight is measured against polystyrene as standard in dimethylacetamide at 30° C.

The polyurethaneureas essential to the invention are prepared by reacting synthesis components which comprise at least one polycarbonate polyol component a), at least one polyisocyanate component b), at least one polyoxyalkylene ether component c), at least one diamine and/or amino alcohol component d) and, if desired, a further polyol component.

Dispersing in water gives the dispersions according to the invention.

The present invention therefore likewise provides a process for preparing the polyurethaneurea dispersions, in which a polycarbonate polyol component a), at least one polyisocyanate component b), at least one polyoxyalkylene ether component c), at least one diamine and/or amino alcohol component d) and, if desired, a further polyol component are reacted with one another and dispersing in water takes place.

Component a) comprises at least one polycarbonate polyol a1), which is obtained by reacting carbonic acid derivatives, such as diphenyl carbonate, dimethyl carbonate or phosgene with difunctional alcohols of the formula (II)

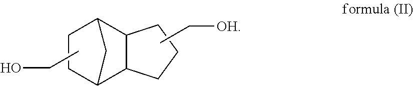

For the preparation in a pressure reactor and at elevated temperature, TCD Alcohol DM [3(4),8(9)-bis(hydroxymethyl)tricyclo(5.2.1.0/2.6)decane/tricyclodecanedimethanol] is reacted with diphenyl carbonate, dimethyl carbonate or phosgene. Reaction with dimethyl carbonate is preferred. Where dimethyl carbonate is used, the methanol elimination product is removed by distillation in a mixture with excess dimethyl carbonate.

These polycarbonate polyols a1) based on diols of the formula (II) have molecular weights, as determined through the OH number, of preferably 200 to 10 000 g/mol, more preferably 300 to 8000 g/mol and very preferably 400 to 6000 g/mol.

Component a) is preferably a mixture of aforementioned polycarbonate polyols a1) based on diols of the formula (II) and further polycarbonate polyols a2).

Such further polycarbonate polyols a2) preferably have average hydroxyl functionalities of 1.7 to 2.3, more preferably of 1.8 to 2.2, very preferably of 1.9 to 2.1.

Furthermore, the polycarbonate polyols a2) have molecular weights, as determined through the OH number, of preferably 400 to 6000 g/mol, more preferably 500 to 5000 g/mol, in particular of 600 to 3000 g/mol, which are obtainable, for example, by reaction of carbonic acid derivatives, such as diphenyl carbonate, dimethyl carbonate or phosgene, with polyols, preferably diols. Suitable such diols include, for example, ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

These polycarbonate polyols a2) contain preferably 40% to 100% by weight of hexanediol, preferably 1,6-hexanediol and/or hexanediol derivatives, preferably those which as well as terminal OH groups have ether groups or ester groups, examples being products obtained by reacting 1 mol of hexanediol with at least 1 mol, preferably 1 to 2 mol of caprolactone or by etherifying hexanediol with itself to give the dihexylene or trihexylene glycol, as synthesis components. Polyether-polycarbonate diols can be used as well. The hydroxyl polycarbonates ought to be substantially linear. Where appropriate, however, they may be slightly branched as a result of the incorporation of polyfunctional components, especially low molecular weight polyols.

Examples of polyols suitable for this purpose include glycerol, hexane-1,2,6-triol, butane-1,2,4-triol, trimethylolpropane, pentaerythritol, quinitol, mannitol, sorbitol, methylglycoside or 1,3,4,6-dianhydrohexitols. Preference is given to such polycarbonates a2) based on hexane-1,6-diol and also on modifying co-diols such as, for example, butane-1,4-diol or else on ε-caprolactone. Further preferred polycarbonate diols a2) are those based on mixtures of hexane-1,6-diol and butane-1,4-diol.

In one preferred embodiment, a mixture is used in a) of the polycarbonate polyols a1) and those polycarbonate polyols a2) based on hexane-1,6-diol, butane-1,4-diol or mixtures thereof.

In the case of mixtures of the constituents a1) and a2), the fraction of a1) as a proportion of the mixture is preferably at least 5 mol %, more preferably at least 10 mol %, based on the total molar amount of polycarbonate.

The polyurethaneureas essential to the invention additionally have units which derive from at least one polyisocyanate as synthesis component b).

As polyisocyanates b) it is possible to use all of the aromatic, araliphatic, aliphatic and cycloaliphatic isocyanates that are known to the person skilled in the art and have an average NCO functionality ≧1, preferably ≧2, individually or in any desired mixtures with one another, it being immaterial whether they have been prepared by phosgene processes or phosgene-free processes. They may also have iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, having a carbon backbone (without the NCO groups present) of 3 to 30, preferably 4 to 20 carbon atoms.

Particularly preferred compounds of component b) correspond to the type mentioned above with aliphatically and/or cycloaliphatically attached NCO groups, such as, for example, bis(isocyanatoalkyl)ethers, bis- and tris(isocyanatoalkyl)benzenes, -toluenes, and -xylenes, propane diisocyanates, butane diisocyanates, pentane diisocyanates, hexane diisocyanates (e.g., hexamethylene diisocyanate, HDI), heptane diisocyanates, octane diisocyanates, nonane diisocyanates (e.g. trimethyl-HDI (TMDI) generally in the form of a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component b) are hexamethylene diisocyanate (HDI), trimethyl-HDI (TMDI), 2-methylpentane-1,5-diisocyanate isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate and/or 4,4′-bis(isocyanatocyclohexyl)methane (H₁₂MDI) or mixtures of these isocyanates. Further examples are derivatives of the aforementioned diisocyanates with uretdione, isocyanurate, urethane, allophanate, biuret, iminooxadiazinedione and/or oxadiazinetrione structure and with more than two NCO groups.

The amount of constituent b) in the preparation of the polyurethaneureas essential to the invention is preferably 1.0 to 3.5 mol, more preferably 1.0 to 3.3 mol, in particular 1.0 to 3.0 mol, based in each case on the amount of the compounds of component a).

The polyurethaneurea used in the present invention has units which derive from a copolymer of polyethylene oxide and polypropylene oxide as synthesis component c). These copolymer units are present in the form of end groups in the polyurethaneurea and have the effect of a particularly advantageous hydrophilicization.

Nonionically hydrophilicizing compounds c) of this kind are, for example, monofunctional polyalkylene oxide polyether alcohols that have on average 5 to 70, preferably 7 to 55, ethylene oxide units per molecule, of the kind obtainable in a manner known per se by alkoxylating suitable starter molecules (e.g. in Ullmanns Enzyklopädie der technischen Chemie, 4th Edition, Volume 19, Verlag Chemie, Weinheim, pp. 31-38).

Suitable starter molecules are, for example, saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and also heterocyclic secondary amines such as morpholine, pyrrolidine, piperidine or 1H-pyrazole. Preferred starter molecules are saturated monoalcohols. Particular preference is given to using diethylene glycol monobutyl ether as starter molecule.

The alkylene oxides, ethylene oxide and propylene oxide, can be used in any order or else in a mixture in the alkoxylation reaction.

The polyalkylene oxide polyether alcohols are mixed polyalkylene oxide polyethers of ethylene oxide and propylene oxide, and preferably at least 30 mol %, more preferably at least 40 mol %, of their alkylene oxide units are composed of ethylene oxide units. Preferred nonionic compounds are monofunctional mixed polyalkylene oxide polyethers which have at least 40 mol % of ethylene oxide units and not more than 60 mol % of propylene oxide units.

The average molar weight of the polyoxyalkylene ether is preferably 500 g/mol to 5000 g/mol, more preferably 1000 g/mol to 4000 g/mol, in particular 1000 to 3000 g/mol.

The amount of constituent c) in the preparation of the polyurethaneureas that are essential to the invention is preferably 0.01 to 0.5 mol, more preferably 0.02 to 0.4 mol, in particular 0.04 to 0.3 mol, based in each case on the amount of the compounds of component a).

In accordance with the invention, it has been possible to show that the polyurethaneureas with end groups which are based on mixed polyoxyalkylene ethers of polyethylene oxide and polypropylene oxide are particularly suitable for producing coatings having a high hydrophilicity.

The polyurethaneureas that are essential to the invention have units which derive from at least one diamine or amino alcohol as a synthesis component, and serve as what are known as chain extenders d).

Such chain extenders are, for example, diamines or polyamines and also hydrazides, examples being hydrazine, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄)-di- and tetraalkyldicyclohexylmethanes, e.g. 4,4′-diamino-3,5-diethyl-3′,5′-diisopropyldicyclohexylmethane.

Suitable diamines or amino alcohols are generally diamines or amino alcohols of low molecular weight which contain active hydrogen whose reactivity towards NCO groups differs, such as compounds which as well as a primary amino group also have secondary amino groups, or as well as an amino group (primary or secondary) also have OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and with particular preference diethanolamine.

Constituent d) of the polyurethaneureas that are essential to the invention can be used as a chain extender in their preparation.

The amount of constituent d) in preparing the polyurethaneureas that are essential to the invention is preferably 0.1 to 1.5 mol, more preferably 0.2 to 1.3 mol, in particular 0.3 to 1.2 mol, based in each case on the amount of the compounds of component a).

In a further embodiment, the polyurethaneureas that are essential to the invention comprise additional units which derive from at least one further polyol as a synthesis component.

The further, low molecular weight polyols e) that are used to synthesize the polyurethaneureas generally have the effect of stiffening and/or of branching of the polymer chain. The molecular weight is preferably 62 to 500 g/mol, more preferably 62 to 400 g/mol, in particular 62 to 200 g/mol.

Suitable polyols may contain aliphatic, alicyclic or aromatic groups. Mention may be made here, for example, of the low molecular weight polyols having up to about 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), and also trimethylolpropane, glycerol or pentaerythritol and mixtures thereof and also, where appropriate, of further low molecular weight polyols. Ester diols can be used as well, such as, for example, α-hydroxybutyl-ε-hydroxycaproic ester, ω-hydroxyhexyl-γ-hydroxybutyric ester, adipic acid β-hydroxyethyl ester or terephthalic acid bis(β-hydroxyethyl) ester.

The amount of constituent e) in preparing the polyurethaneureas that are essential to the invention is, if present, preferably 0.05 to 1.0 mol, more preferably 0.05 to 0.5 mol, in particular 0.1 to 0.5 mol, based in each case on the amount of the compounds of component a).

The reaction of the isocyanate-containing component b) with the hydroxy- or amine-functional compounds a), c), d) and, where appropriate, e) is typically accomplished while observing a slight NCO excess over the reactive hydroxy or amine compounds. These residues must be broken down or blocked so that there is no reaction with large polymer chains. Such reaction leads to the three-dimensional crosslinking and gelling of the batch, and so further processing is no longer possible.

Customarily, however, the excess isocyanate groups are hydrolysed and broken down by the dispersing water during the dispersing step.

If the residual isocyanate content has been blocked during the preparation of the polyurethaneureas that are essential to the invention, they also have, as synthesis components, monomers f) which are in each case located at the chain ends and cap them.

These synthesis components derive on the one hand from monofunctional compounds that are reactive with NCO groups, such as monoamines, especially mono-secondary amines, or monoalcohols. Examples that may be mentioned here include ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the building blocks f) are used in the polyurethaneurea solutions according to the invention essentially in order to destroy the NCO excess, the amount required is dependent essentially on the amount of the NCO excess, and cannot be specified in general terms.

In one preferred embodiment of the present invention no component f) is used, and so the polyurethaneurea that is essential to the invention comprises only the constituents a) to d) and, if desired, component e).

In the preparation of the polyurethaneureas that are essential to the invention, the synthesis components described in more detail above are generally reacted so as first to prepare an isocyanate-functional prepolymer that is free from urea groups, by reaction of constituents a), b), c) and, where appropriate, e), the amount-of-substance ratio of isocyanate groups to isocyanate-reactive groups being preferably 0.8 to 4.0, more preferably 0.9 to 3.8, in particular from 1.0 to 3.5.

In an alternative embodiment it is also possible first to react constituent a) separately with the isocyanate b). After that, then, constituents c) and e) can be added and reacted. Subsequently, in general, the isocyanate groups that have remained are subjected to amino-functional chain extension or termination before, during or after dispersing in water, the equivalent ratio of isocyanate-reactive groups of the compounds used for chain extending to free isocyanate groups of the prepolymer being preferably between 40% to 150%, more preferably between 50% to 120%, in particular between 60% to 120% (constituent d)).

In this context the polyurethane dispersions of the invention are prepared preferably by the process known as the acetone process. For the preparation of the polyurethane dispersion by this acetone process, customarily constituents a), c) and e), which must contain no primary or secondary amino groups, and the polyisocyanate component b), for preparing an isocyanate-functional polyurethane prepolymer, are introduced in whole or in part as an initial charge and where appropriate are diluted with a solvent which is miscible with water but inert toward isocyanate groups, and the diluted or undiluted initial charge is heated to temperatures in the range from 50 to 120° C. The isocyanate addition reaction can be accelerated by using the catalysts that are known in polyurethane chemistry, an example being dibutyltin dilaurate. Synthesis without catalyst is preferred.

Suitable solvents are the customary aliphatic, keto-functional solvents such as, for example, acetone, butanone, which can be added not only at the beginning of the preparation but also, where appropriate, in portions later on as well. Acetone and butanone are preferred. Other solvents such as, for example, xylene, toluene, cyclohexane, butyl acetate, methoxypropyl acetate, solvents with ether units or ester units may likewise be employed and may be distilled off in whole or in part or may remain completely in the dispersion.

Subsequently the constituents of c) and e) not yet added, if appropriate, at the beginning of the reaction are metered in.

Preferably the prepolymer is prepared without addition of solvent, and for chain extension only is diluted with a suitable solvent, preferably acetone.

The reaction to the prepolymer takes place partly or completely, but preferably completely. In this way polyurethane prepolymers which contain free isocyanate groups are obtained, in bulk or in solution.

Subsequently, in a further process step, if it has not already taken place or has taken place only partly, the prepolymer obtained is dissolved using aliphatic ketones such as acetone or butanone.

Subsequently, possible NH₂—, NH-functional and/or OH-functional components are reacted with the remaining isocyanate groups. This chain extension/chain termination may be carried out either in solvent prior to dispersing, during dispersing, or in water after dispersing. The chain extension is preferably carried out prior to dispersing in water.

Where compounds meeting the definition of d) with NH₂ groups or NH groups are used for the chain extension, the chain extension of the prepolymers takes place preferably prior to dispersing.

The degree of chain extension, in other words the equivalent ratio of NCO-reactive groups of the compounds used for chain extension to free NCO groups of the prepolymer, is preferably between 40% to 150%, more preferably between 50% to 120%, in particular between 60% to 120%.

The aminic components d) may be used where appropriate in water- or solvent-diluted form in the process of the invention individually or in mixtures, with any sequence of addition being possible in principle.

If water or organic solvents are used as diluents, the diluent content is preferably 70% to 95% by weight.

The preparation of the polyurethane dispersion from the prepolymers takes place following chain extension. For that purpose, the dissolved and chain-extended polyurethane polymer is introduced into the dispersing water, where appropriate with strong shearing, such as vigorous stirring, for example, or else, conversely, the dispersing water is added to the prepolymer solutions with stirring. Preferably the water is added to the dissolved prepolymer.

The solvent still present in the dispersions after the dispersing step is usually then removed distillatively. Removal during the dispersing procedure itself is likewise possible.

The solids content of the polyurethane dispersion after synthesis is in the range from 20% to 70% by weight, preferably 20% to 65% by weight. For coating experiments, these dispersions can be diluted arbitrarily with water in order to allow variable adjustment of the thickness of the coating. All concentrations from 1% to 60% by weight are possible; concentrations in the 1% to 40% by weight range are preferred.

In this context it is possible to achieve any desired coat thicknesses, such as for example from a few 100 nm up to a few 100 μm, with higher and lower thicknesses also being possible in the context of the present invention.

The polyurethaneurea dispersions of the invention may further comprise additives and constituents that are customary for the particular end use intended.

One example of such are pharmacological actives, medicaments and additives which promote the release of pharmacological actives (“drug-eluting additives”).

Pharmacological actives and medicaments which can be used in the coatings of the invention on the medical devices and which therefore may be present in the solutions according to the invention are, for example, thromboresistant agents, antibiotic agents, anti-tumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts and combinations thereof.

Specific examples of such pharmacological actives and medicaments hence include thromboresistant (non-thrombogenic) agents and other agents for suppressing an acute thrombosis, stenosis or late re-stenosis of the arteries, examples being heparin, streptokinase, urokinase, tissue plasminogen activator, anti-thromboxan-B₂ agent; anti-B-thromoboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor likewise may be utilized as a medicament in order to suppress subintimal fibromuscular hyperplasia at the arterial stenosis site, or any other cell growth inhibitor can be utilized at the stenosis site.

The pharmacological active or medicament may also be composed of a vasodilator, in order to counteract vasospasm—for example, an antispasm agent such as papaverine. The medicament may be a vasoactive agent per se, such as calcium antagonists, or α- and β-adrenergic agonists or antagonists. In addition the therapeutic agent may be a biological adhesive such as cyanoacrylate in medical grade or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include the following:
a) heparin, heparin sulphate, hirudin, hyaluronic acid, chondroitin sulphate, dermatan sulphate, keratan sulphate, lytic agents, including urokinase and streptokinase, their homologues, analogues, fragments, derivatives and pharmaceutical salts thereof;
b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;
c) paclitaxel, docetaxel, immunosuppressants such as sirolimus or everolimus, alkylating agents, including mechlorethamine, chlorambucil, cyclophosphamide, melphalane and Ifosfamide; antimetabolites, including methotrexate, 6-mercaptopurine, 5-fluorouracil and cytarabine; plant alkoids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof;
d) antiviral agents such as amantadine, rimantadine, rabavirin, idoxuridine, vidarabin, trifluridine, acyclovir, ganciclorir, zidovudine, phosphonoformates, interferons, their homologues, analogues, fragments, derivatives, pharmaceutical salts and mixtures thereof; and
e) antiinflammatory agents such as, for example, ibuprofen, dexamethasone or methylprednisolone.

To generate surfaces having infestation-inhibiting properties, the coating compositions of the invention may comprise the active infestation inhibitors known from the prior art. Their presence generally boosts the already outstanding infestation-inhibiting properties of the surfaces produced with the coating compositions of the invention themselves.

Further additions such as, for example, antioxidants, pigments hand agents, dyes, matting agents, UV stabilizers, light stabilizers, hydrophobicizers, buffering substances, flow control assistants and/or thickeners for viscosity adjustment are used.

The polyurethaneurea dispersions of the invention can be used to form a coating for example on a medical device.

The term “medical device” is to be understood broadly in the context of the present invention. Suitable, non-limiting examples of medical devices (including instruments) are contact lenses; cannulas; catheters, for example urological catheters such as urinary catheters or ureteral catheters; central venous catheters; venous catheters or inlet or outlet catheters; dilation balloons; catheters for angioplasty and biopsy; catheters used for introducing a stent, an embolism filter or a vena caval filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires.

The dispersions according to the invention may be used, furthermore, for producing protective coatings, for example for gloves, stents and other implants; external (extracorporeal) blood lines (blood-carrying tubes); membranes; for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

Coatings on the basis of the dispersions of the invention are particularly advantageous for medical applications specifically on account of the fact that they contain no organic solvent residues and hence are in general toxically unobjectionable, and at the same time lead to a more pronounced hydrophilicity, which is evident, for example, from a low contact angle.

In addition to the hydrophilic properties of improving the lubricity, the coating compositions provided in accordance with the invention are also notable for a high level of blood compatibility. This makes working with these coatings advantageous in blood contact in particular. The materials exhibit reduced coagulation tendency in blood contact as compared with polymers of the prior art.

Systems which release actives and are based on the hydrophilic coating materials of the invention are also conceivable outside medical technology, such as for example for applications in crop protection as a carrier material for actives. The entire coating may in that case be considered an active-releasing system and may be used, for example, to coat seed (seed grains). As a result of the hydrophilic properties of the coating, the active it contains is able to emerge in the moist earth and develop its intended effect, without adversely affecting the capacity of the seed to germinate. In the dry state, however, the coating composition binds the active securely to the seed, and so, for example, the active is not detached when the seed grain is being fired into the soil by the broadcasting machine; as a result of such detachment, the active could develop unwanted effects, for example, on the fauna that are present (jeopardizing bees by insecticides intended per se to prevent the attack of insects on the seed grain in the soil).

Beyond their application as a coating for medical devices, the polyurethane dispersions according to the invention can also be utilized for further technical applications in the non-medical sector.

Thus, the polyurethane dispersions according to the invention serve for producing coatings as protection of surfaces against fogging with moisture, for the production of easy-to-clean or self-cleaning surfaces. These hydrophilic coatings also reduce the pick-up of dirt and prevent the formation of water spots. Conceivable applications in the exterior sector are, for example, windows and roof lights, glass facades or Plexiglas roofs. In the interior sector, materials of this kind can be utilized for the coating of surfaces of sanitary equipment. Further applications are the coating of spectacle lenses or of packaging materials such as food packaging for the purpose of preventing moisture fogging or droplet formation due to condensed water.

The polyurethane dispersions according to the invention are also suitable for treating surfaces in contact with water for the purpose of reducing infestation. This effect is also referred to as the antifouling effect. One very important application of this antifouling effect is in the area of the underwater coatings on ships' hulls. Ships' hulls without an antifouling treatment very quickly become infested by marine organisms, leading to increased friction and hence to a reduction in the possible speed and a higher consumption of fuel. The coating materials of the invention reduce or prevent infestation by marine organisms, and prevent the above-described disadvantages of this infestation. Further applications in the area of antifouling coatings are articles for fishing such as fishing-nets and also all metallic substrates in underwater use, such as pipelines, offshore drilling platforms, locks and lock gates, etc. Hulls which have surfaces generated with the coating materials of the invention, especially below the water line, also possess a reduced frictional resistance, and so ships thus equipped either have a reduced fuel consumption or achieve higher speeds. This is of interest in particular in the sporting boat sector and in yacht building.

A further important field for application of the abovementioned hydrophilic coating materials is the printing industry. By means of the coatings of the invention, hydrophobic surfaces can be made hydrophilic and as a result can be printed with polar printing inks, or can be printed using ink jet technology.

A further field for application of the hydrophilic coatings of the invention is in formulations for cosmetic applications.

Coatings of the polyurethane dispersions according to the invention can be applied by means of a variety of methods. Examples of suitable coating techniques for these dispersions include knife coating, printing, transfer coating, spraying, spin coating or dipping.

A wide variety of substrates can be coated, such as metals, textiles, ceramics and plastics. Preference is given to coating medical devices manufactured from plastic or metal. Examples of metals that can be mentioned include the following: medical stainless steel and nickel titanium alloys. Many polymer materials are conceivable from which the medical devices may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethanes to the medical device, further suitable coatings may be applied as a base before these hydrophilic coating materials are applied.

### EXAMPLES

The NCO content of the resins described in the inventive and comparative examples was determined by titration in accordance with DIN EN ISO 11909.

The solids contents were determined in accordance with DIN-EN ISO 3251. Polyurethane dispersion (1 g) was dried at 115° C. to constant weight (15-20 min) using an infrared drier.

The average particle sizes of the polyurethane dispersions were measured using the High Performance Particle Sizer (HPPS 3.3) from Malvern Instruments.

The tensile strengths were determined in accordance with DIN 53504.

Unless noted otherwise, the amounts reported in % are to be understood as % by weight and relate to the aqueous dispersion obtained.

### Substances Used and Abbreviations:

Desmophen C2200: polycarbonate polyol, OH number 56 mg KOH/g, number-average molecular weight 2000 g/mol (Bayer MaterialScience Ag, Leverkusen, De)
Desmophen C1200: polycarbonate polyol, OH number 56 mg KOH/g, number-average molecular weight 2000 g/mol (Bayer MaterialScience Ag, Leverkusen, De)
Desmophen XP 2613 polycarbonate polyol, OH number 56 mg KOH/g, number-average molecular weight 2000 g/mol (Bayer MaterialScience Ag, Leverkusen, De)
Polyether LB 25: monofunctional polyether based on ethylene oxide/propylene oxide, number-average molecular weight 2250 g/mol, OH number 25 mg KOH/g (Bayer MaterialScience Ag, Leverkusen, De)
TCD Alcohol DM 3 (4),8(9)-bis(hydroxymethyl)tricyclo(5.2.1.0/2.6) decane/tricyclodecanedimethanol from Celanese Chemicals, Dallas, USA

### Example 1

### Preparation of a Cycloaliphatic Polycarbonate Diol Based on TCD Alcohol DM with a Number-Average Molecular Weight of 1300 g/mol

A 16 1 pressure reactor with top-mounted distillation attachment, stirrer and receiver was charged with 5436 g of TCD Alcohol DM along with 1.2 g of yttrium(III) acetylacetonate and also 3810 g of dimethyl carbonate at 80° C. Subsequently, under a nitrogen atmosphere, the reaction mixture was heated to 135° C. over 2 h and maintained there with stirring for 24 h, during which the pressure climbed to 6.3 bar (absolute). It was then cooled to 60° C. and air was admitted. The methanol elimination product was then removed by distillation in a mixture with dimethyl carbonate, the temperature being raised in steps to 150° C. The mixture was then stirred at 150° C. for a further 4 hours, subsequently heated to 180° C., and then stirred at 180° C. for a further 4 h. The temperature was then reduced to 90° C. and a stream of nitrogen (5 l/h) was passed through the reaction mixture, during which the pressure was lowered to 20 mbar. Thereafter the temperature was increased to 180° C. over 4 h and held there for 6 h. In the course of this operation, methanol was removed further from the reaction mixture, in a mixture with dimethyl carbonate.

After air had been admitted and the reaction mixture cooled to room temperature, a yellowish solid polycarbonate diol was obtained that had the following characteristics: Mₙ=1290 g/mol; OH number=87 mg KOH/g

### Example 2

### Preparation of a Cycloaliphatic Polycarbonate Diol Based on TCD Alcohol DM with a Number-Average Molecular Weight of about 500 g/mol

Procedure as in Example 1, using 7790 g of TCD Alcohol DM, 1.68 g of yttrium(III) acetylacetonate and 3096 g of dimethyl carbonate.

This gave a yellowish polycarbonate diol of high viscosity that had the following characteristics: Mₙ=496 g/mol; OH number=226 mg KOH/g; viscosity at 75° C.=138 400 mPas.

### Example 3

### Preparation of a (Cyclo)Aliphatic Polycarbonate Diol Based on TCD Alcohol DM and 1,4-Butanediol with a Number-Average Molecular Weight of About 1000 g/mol

Procedure as in Example 1, using 5951 g of TCD Alcohol DM, 2732 g of 1,4-butanediol, 2.0 g of yttrium(III) acetylacetonate and 6842 g of dimethyl carbonate.

This gave a colourless polycarbonate diol that had the following characteristics: Mₙ=943 g/mol; OH number=119 mg KOH/g; viscosity at 75° C.=15 130 mPas.

### Example 4

### Comparative

277.2 g of Desmophen C 2200, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanato-cyclohexyl)methane (H₁₂MDI) and then of 11.9 g of isophorone diisocyanate. The mixture was heated to 110° C. After 16 hours the theoretical NCO value of 2.4% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 40.7% and an average particle size of 136 nm.

### Example 5

### Inventive

208.0 g of Desmophen C 2200, 45.2 g of polycarbonate diol of Example 1, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexypmethane (H₁₂MDI) and then of 11.9 g of isophorone diisocyanate. The mixture was heated to 110° C. After 16 hours the theoretical NCO value of 2.6% was reached. The completed prepolymer was dissolved in 700 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 550 g of water. The solvent was removed by distillation under reduced pressure. This gave a storage-stable polyurethane dispersion having a solids content of 39.0% and an average particle size of 131 nm.

### Example 6

### Inventive

138.6 g of Desmophen C 2200, 90.1 g of polycarbonate diol of Example 1, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 2 hours 45 minutes the theoretical NCO value of 2.8% was reached. The completed prepolymer was dissolved in 700 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 550 g of water. The solvent was removed by distillation under reduced pressure. This gave a storage-stable polyurethane dispersion having a solids content of 39.6% and an average particle size of 157 nm.

### Example 7

### Inventive

184.8 g of Desmophen C 2200, 23.1 g of polycarbonate diol of Example 2, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 21 hours the theoretical NCO value of 2.9% was reached. The completed prepolymer was dissolved in 650 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 490 g of water. The solvent was removed by distillation under reduced pressure. This gave a storage-stable polyurethane dispersion having a solids content of 40.3% and an average particle size of 117 nm.

### Example 8

### Inventive

138.6 g of Desmophen C 2200, 34.7 g of polycarbonate diol of Example 2, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 18 hours the theoretical NCO value of 3.3% was reached. The completed prepolymer was dissolved in 650 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 450 g of water. The solvent was removed by distillation under reduced pressure. This gave a storage-stable polyurethane dispersion having a solids content of 40.5% and an average particle size of 151 nm.

### Example 9

### Inventive

138.6 g of Desmophen C 2200, 69.3 g of polycarbonate diol of Example 3, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 2 hours 15 minutes the theoretical NCO value of 2.9% was reached. The completed prepolymer was dissolved in 650 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 520 g of water. The solvent was removed by distillation under reduced pressure. This gave a storage-stable polyurethane dispersion having a solids content of 38.0% and an average particle size of 190 nm.

### Example 10

### Contact Angles and 100% Moduli of Comparative Example 4 Versus Inventive Examples 5-9

### 1. Production of the Coatings for the Measurement of the Static Contact Angle

The coatings for the measurement of the static contact angle were produced on glass slides measuring 25×75 mm using a spin coater (RC5 Gyrset 5, Karl Stiss, Garching, Germany). For this purpose, a slide was clamped in on the sample plate of the spin coater and covered homogeneously with about 2.5-3 g of aqueous undiluted polyurethane dispersion. Rotation of the sample plate at 1300 revolutions per minute for 20 seconds gave a homogeneous coating, which was dried at 100° C. for 15 min and then at 50° C. for 24 h. The coated slides obtained were subjected directly to a contact angle measurement.

A static contact angle measurement is performed on the resulting coatings on the slides. Using the video contact angle measuring instrument OCA20 from Dataphysics, with computer-controlled injection, 10 drops of Millipore water are applied to the specimen, and their static wetting angle is measured. Beforehand, using an antistatic drier, the static charge (if present) on the sample surface is removed.

### 2. Production of the Coatings for the Measurement of the 100% Modulus

Films are produced on release paper using a 200 μm doctor blade, and are dried at 100° C. for 15 minutes. This is followed by drying at 100° C. for 15 minutes. Punched shapes are investigated in accordance with DIN 53504.

The coatings were applied to release paper using a 200 μm doctor blade. Prior to film production, the aqueous dispersions are admixed with 2% by weight of a thickener (Borchi Gel A LA, Borchers, Langenfeld, Germany) and homogenized by stirring at RT for 30 minutes. The wet films were dried at 100° C. for 15 minutes.

The investigations were carried out in accordance with DIN 53504.

### 3. Results of Investigation

TABLE 1
Contact angles and 100% moduli of the films from materials of Examples 4-9

| Example No. | Contact angle (°) | 100% modulus (N/mm²) |
|---|---|---|
| Comparative | 10 | 2.6 |
| Example 4 | | |
| Example 5 | 12 | 3.0 |
| Example 6 | 17 | 6.7 |
| Example 7 | 16 | 3.6 |
| Example 8 | 27 | 7.2 |
| Example 9 | 16 | 5.4 |

Inventive Examples 5 to 9 differ in that, in comparison to comparative Example 4, some of the polycarbonate diol Desmophen C2200 was replaced by a new polycarbonate diol of the invention. In the form of a coating, the materials have hydrophilic properties similar to those of comparative Example 4, always contact angles smaller than 30°. The 100% moduli are all higher than that of comparative Example 4.

### Example 11

### Comparative

282.1 g of Desmophen C 2200, 22.0 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanato-cyclohexyl)methane (H₁₂MDI) and then of 11.9 g of isophorone diisocyanate. The mixture was heated to 110° C. After 21.5 hours the theoretical NCO value of 2.4% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 41.7% and an average particle size of 207 nm.

### Example 12

### Inventive

141.2 g of Desmophen C 2200, 35.3 g of polycarbonate diol of Example 2, 22.0 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 18 hours the theoretical NCO value of 3.4% was reached. The completed prepolymer was dissolved in 600 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 400 g of water. The solvent was removed by distillation under reduced pressure. This gave a storage-stable polyurethane dispersion having a solids content of 41.6% and an average particle size of 219 nm.

### Example 13

### Contact Angles and 100% Moduli of Comparative Example 11 Versus Inventive Example 12

The production of the coatings and also the determination of the contact angles and 100% moduli take place as described in Example 10.

TABLE 2
Contact angles and 100% moduli of the films
of materials of Examples 12 and 13

| Example No. | Contact angle (°) | 100% modulus (N/mm²) |
|---|---|---|
| Comparative | 24 | 3.3 |
| Example 11 | | |
| Example 12 | 36 | 9.2 |

In comparison to comparative Example 11, inventive Example 12 includes fractions of a polycarbonate diol of the invention. The surface of the coating continues to be very hydrophilic, while the 100% modulus goes up by almost three times.

### Example 14

### Comparative

282.1 g of Desmophen XP 2613, 22.0 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanato-cyclohexyl)methane (H₁₂MDI) and then of 11.9 g of isophorone diisocyanate. The mixture was heated to 110° C. After 70 minutes the theoretical NCO value of 2.5% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 38.3% and an average particle size of 215 nm.

### Example 15

### Inventive

141.2 g of Desmophen XP 2613, 91.8 g of polycarbonate diol of Example 1, 22.0 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 60 minutes the theoretical NCO value was reached. The completed prepolymer was dissolved in 650 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 530 g of water. The solvent was removed by distillation under reduced pressure. This gave a storage-stable polyurethane dispersion having a solids content of 38.2% and an average particle size of 327 nm.

### Example 16

### Contact Angles and 100% Moduli of Comparative Example 14 Versus Inventive Example 15

The production of the coatings and also the determination of the contact angles and 100% moduli take place as described in Example 10.

TABLE 3
Contact angles and 100% moduli of the
films of materials from Examples 14 and 15

| Example No. | Contact angle (°) | 100% modulus (N/mm²) |
|---|---|---|
| Comparative | 41 | 3.0 |
| Example 14 | | |
| Example 15 | 41 | 12.3 |

In comparison to comparative Example 14, inventive Example 15 includes fractions of a polycarbonate diol of the invention. The contact angle of the coating is changed hardly at all, while the 100% modulus goes up by four times.

### Example 17

### Comparative

269.8 g of Desmophen C 2200, 49.7 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanato-cyclohexyl)methane (H₁₂MDI) and then of 11.9 g of isophorone diisocyanate. The mixture was heated to 110° C. After 21 hours the theoretical NCO value of 2.4% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 41.3% and an average particle size of 109 nm.

### Example 18

### Inventive

135.0 g of Desmophen C 2200, 33.8 g of polycarbonate diol of Example 2, 49.7 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 20 hours the theoretical NCO value was reached. The completed prepolymer was dissolved in 590 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 33.7% and an average particle size of 83 nm.

### Example 19

### Contact Angles and 100% Moduli of Comparative Example 17 Versus Inventive Example 18

The production of the coatings and also the determination of the contact angles and 100% moduli take place as described in Example 10.

TABLE 4
Contact angles and 100% moduli of the
films of materials from Examples 17 and 18

| Example No. | Contact angle (°) | 100% modulus (N/mm²) |
|---|---|---|
| Comparative | 11 | 1.9 |
| Example 17 | | |
| Example 18 | 9 | 6.0 |

In comparison to comparative Example 18, inventive Example 17 includes fractions of a polycarbonate diol of the invention. The contact angle of the coating is changed hardly at all, while the 100% modulus goes up by three times.

### Example 20

### Comparative

277.2 g of Desmophen C 2200, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanato-cyclohexyl)methane (H₁₂MDI) and then of 11.9 g of isophorone diisocyanate. The mixture was heated to 110° C. After 75 minutes the theoretical NCO value of 2.4% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 590 g of water. The solvent was removed by distillation under reduced pressure. This gave a storage-stable polyurethane dispersion having a solids content of 39.9% and an average particle size of 169 nm

### Example 21

### Inventive

138.6 g of Desmophen C 2200, 34.7 g of polycarbonate diol of Example 2, 33.1 g of Polyether LB 25 and 6.7 g of neopentyl glycol were introduced at 65° C. and homogenized by stirring for 5 minutes. This mixture was admixed by the addition at 65° C., over the course of 1 minute, first of 71.3 g of 4,4′-bis(isocyanatocyclohexyl)methane (H₁₂MDI) and then of 11.9 g of isophorone diisocyanate. The mixture was heated to 110° C. After 75 minutes the theoretical NCO value was reached. The completed prepolymer was dissolved in 650 g of acetone at 50° C. and then at 40° C. a solution of 4.8 g of ethylenediamine in 16 g of water was metered in over the course of 10 minutes. The subsequent stirring time was 5 minutes. After that, over the course of 15 minutes, dispersion was carried out by addition of 450 g of water. The solvent was removed by distillation under reduced pressure. This gave a storage-stable polyurethane dispersion having a solids content of 40.0% and an average particle size of 167 nm.

### Example 22

### Contact Angles and 100% Moduli of Comparative Example 20 Versus Inventive Example 21

The production of the coatings and also the determination of the contact angles and 100% moduli take place as described in Example 10.

TABLE 5
Contact angles and 100% moduli of the
films of materials from Examples 20 and 21

| Example No. | Contact angle (°) | 100% modulus (N/mm²) |
|---|---|---|
| Comparative | 14 | 1.6 |
| Example 20 | | |
| Example 21 | 16 | 6.1 |

In comparison to comparative Example 20, inventive Example 21 includes fractions of a polycarbonate diol of the invention. The contact angle of the coating is changed hardly at all, while the 100% modulus goes up by almost four times.

1-13. (canceled)

14. A polyurethaneurea dispersion comprising a polyurethaneurea which is terminated with at least one polyethylene oxide- and polypropylene oxide-based copolymer unit, and comprises a polycarbonate polyol-based unit of formula (I)

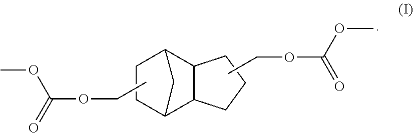

15. The polyurethaneurea dispersion according to claim 14, wherein the polyurethaneurea is free from ionic or ionogenic groups.

16. The polyurethaneurea dispersion according to claim 14, wherein the polyurethaneurea is based on a polycarbonate polyol component.

17. The polyurethaneurea dispersion according to claim 16, wherein the polycarbonate polyol component has an average hydroxyl functionality of 1.7 to 2.3.

18. The polyurethaneurea dispersion according to claim 17, wherein the polycarbonate polyol component comprises a first polycarbonate polyol which is obtained by reacting a carbonic acid derivative with a difunctional alcohol of the formula (II)

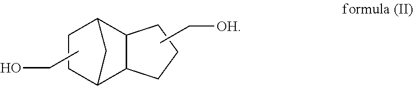

19. The polyurethaneurea dispersions according to claim 18, wherein the polycarbonate polyol component further comprises a second polycarbonate polyol.

20. The polyurethaneurea dispersion according to claim 19, wherein the second polycarbonate polyol is a compound having an average hydroxyl functionality of 1.7 to 2.3 and a molecular weight, as determined through the OH number, of 400 to 6000 g/mol, based on hexane-1,6-diol, butane-1,4-diol, or mixtures thereof.

21. The polyurethaneurea dispersion according to claim 20, wherein the polyurethaneurea is free from ionic or ionogenic groups.

22. The polyurethaneurea dispersion according to claim 14, wherein the copolymer unit of polyethylene oxide and polypropylene oxide is based on a monohydroxy-functional mixed polyalkylene oxide polyether comprising at least 40 mol % ethylene oxide units and not more than 60 mol % propylene oxide units, based on the total fraction of alkylene oxide units, and wherein the monohydroxy-functional mixed polyalkylene oxide polyether has a number-average molecular weight of 500 to 5000 g/mol.

23. The polyurethaneurea dispersion according to claim 14, wherein the polyurethaneurea has a number-average molecular weight of 5000 to 100,000 g/mol as measured in dimethylacetamide at 30° C.

24. The polyurethaneurea dispersion according to claim 14, wherein the polyurethaneurea dispersion further comprises a pharmacological active compound.

25. The polyurethaneurea dispersion according to claim 21,
wherein the polyurethaneurea dispersion further comprises a pharmacological active compound;
wherein the copolymer unit of polyethylene oxide and polypropylene oxide is based on a monohydroxy-functional mixed polyalkylene oxide polyether comprising at least 40 mol % ethylene oxide units and not more than 60 mol % propylene oxide units, based on the total fraction of alkylene oxide units, and wherein the monohydroxy-functional mixed polyalkylene oxide polyether has a number-average molecular weight of 500 to 5000 g/mol; and
wherein the polyurethaneurea has a number-average molecular weight of 5000 to 100,000 g/mol as measured in dimethylacetamide at 30° C.

26. A process for preparing the polyurethaneurea dispersion according to claim 14, which comprises
a. reacting a composition comprising
i. a polycarbonate polyol component,
ii. at least one polyisocyanate component,
iii. at least one polyoxyalkylene ether component,
iv. at least one diamine and/or amino alcohol component, and
v. optionally, a further polyol component; and
b. dispersing the composition in water.

27. A polyurethaneurea obtained from the polyurethaneurea dispersion according to claim 14.

28. A coating obtained with the polyurethaneurea according to claim 27.

29. A substrate coated with the coating according to claim 28.
